(19) United States
(12) Patent Application Publication
(10) Pub. No.: US 2014/0065087 A1
(43) Pub. Date: Mar. 6, 2014

(54) Preservative Mixtures and Polymer Solutions Stabilized Therewith

(71) Applicant: BASF SE, LUDWIGSHAFEN (DE)

(72) Inventor: Bernd de Potzolli, Bad Duerkheim (DE)

(73) Assignee: BASF SE, LUDWIGSHAFEN (DE)

(21) Appl. No.: 13/971,286

(22) Filed: Aug. 20, 2013

Related U.S. Application Data
(60) Provisional application No. 61/693,796, filed on Aug. 28, 2012.

(51) Int. Cl. A61K 8/87; A61Q 5/06; A61K 8/81; A61Q 19/00

(52) U.S. Cl. CPC A61K 8/87; A61Q 19/00; A61Q 5/06; A61K 8/8182
USPC 424/63; 424/78.02; 424/78.03; 424/70.11; 524/83; 514/372

(57) ABSTRACT

Aqueous polymer solutions are described which have been stabilized with a preservative mixture of 0.01 to 0.2% by weight of ethylhexylglycerol, 0.001 to 0.01% by weight of 2-methylisothiazolinone and 0.05 to 0.5% by weight of phenoxyethanol. The stabilized polymer solutions are suitable for cosmetic preparations, such as skincare and hair care compositions, products of decorative cosmetics and cosmetic cleaning products.

### FIELD

The present invention relates to specific biocidal preservative mixtures and to aqueous polymer solutions which have been stabilized with these preservative mixtures or by mixing in said biocidally effective individual components and to their use in cosmetic preparations, such as skincare and hair care compositions, and also hairstyling products.

### BACKGROUND

The growth of microorganisms such as bacteria and viruses and also fungi and yeasts in polymer solutions, in particular aqueous polymer solutions, is to be avoided in many respects. Microorganisms in aqueous polymer solutions lead to an buildup of germs unhealthy and therefore constitute a high hygiene risk, especially if the polymer solutions are incorporated into cosmetic products, for example as binders, thickeners or film formers.

It is known to stabilize aqueous polymer solutions with biocides as preservatives to combat troublesome buildups of germs. They are incorporated into the polymer solution as additives. However, it has been found that some of the known preservatives are carcinogenic and are not suitable for use in cosmetic preparations. Further disadvantages of these additives are that a lack of product stability is to be noted. The result is therefore very often discolorations, clouding, phase separation, negative changes in odor and inadequate microbiological effectiveness in the polymer solutions admixed therewith.

### SUMMARY

It is therefore an object of the present invention to avoid undesired buildups of germs in aqueous polymer solutions while retaining good product stabilities. It is furthermore an object of the present invention to develop a technology with which cosmetic products can be produced without concern as regards their ingredients.

This object has been achieved with the present invention.

### DETAILED DESCRIPTION

The present invention relates to aqueous polymer solutions stabilized with a mixture of preservatives and a booster (subsequently called “preservative mixture”) of
0.01 to 0.2% by weight of ethylhexylglycerol
0.001 to 0.01% by weight of 2-methylisothiazolinone
0.05 to 0.5% by weight of phenoxyethanol.

The present invention likewise provides the polymers present/obtained in/from the stabilized aqueous polymer solutions.

The invention further relates to the use of the aqueous polymer solution in cosmetic preparations, such as skincare and hair care compositions, hairstyling products, products of decorative cosmetics and cosmetic cleaning, e.g. shampoos and shower gels, and also liquid soaps.

Moreover, the invention relates to preservative mixtures for stabilizing aqueous polymer solutions.

The dependent claims relate to preferred embodiments of the aqueous polymer solution according to the invention, to its polymers, and to the use.

The amounts for the constituents of the preservative mixture refer to 100% by weight of aqueous polymer solution.

The aqueous polymer solution according to the invention comprises, in a balanced low dosage, the constituents ethylhexylglycerol, 2-methylisothiazolinone and phenoxyethanol as biocidal active ingredients. These specific preservative mixtures exhibit, in a low concentration range, unexpected effects for the microbial stabilization of the aqueous polymer solutions according to the invention. The synergistically intensifying effectiveness is directed against various germs or germ mixtures, such as, for example, Gram-positive and Gram-negative bacteria, yeasts and molds. In this connection, the product stability of the aqueous polymer solutions according to the invention is retained. This result was unforeseeable, especially in view of the fact that the mixture is present in the polymer solutions in extremely low concentrations.

According to the invention, the aqueous polymer solution comprises, for the stabilization, a preservative mixture of the constituents ethylhexylglycerol, 2-methylisothiazolinone and phenoxyethanol in the following amounts:

0.01 to 0.2% by weight of ethylhexylglycerol,
0.001 to 0.01% by weight of 2-methylisothiazolinone, and
0.05 to 0.5% by weight of phenoxyethanol,
based on 100% by weight of aqueous polymer solution.

A preferred aqueous polymer solution comprises:

0.04 to 0.15% by weight of ethylhexylglycerol,
0.003 to 0.008% by weight of 2-methylisothiazolinone, and
0.1 to 0.5% by weight of phenoxyethanol.

A very particularly preferred aqueous polymer solution comprises

0.04±0.004% by weight of ethylhexylglycerol,
0.0038±0.0004% by weight of 2-methylisothiazolinone, and
0.5±0.05% by weight of phenoxyethanol.

The present invention likewise describes preservative mixtures which are used for the stabilization of aqueous polymer solutions.

A preservative mixture according to the invention (mixture A) has the following constituents as biocidal active ingredients:

62.5% by weight of phenoxyethanol,
25.0% by weight of ethylhexylglycerol,
1.25% by weight of 2-methylisothiazolinone, and
water as the remainder,
based on 100% by weight of mixture.

A further preservative mixture according to the invention (mixture B) has the following composition:

86.20% by weight of phenoxyethanol
6.9% by weight of ethylhexylglycerol and
0.69% by weight of 2-methylisothiazolinone
water as the remainder,
based on 100% by weight of mixture.

The aqueous polymer solution of the present invention is microbially stabilized by the specific preservative mixtures. In order to achieve the claimed concentration range in the aqueous polymer solution, the mixture A is used in an amount of from 0.08 to 0.8% by weight. The mixture A is used in an amount of from 0.2 to 0.6% by weight in order to obtain the preferred aqueous polymer solution. The mixture B is used in an amount of from 0.4 to 0.6% by weight in order to prepare the very particularly preferred aqueous polymer solution.

The term “polymer” comprises for example linear, water-soluble branched or water-insoluble linear and branched polymers. The term “water-insoluble branched polymer” also comprises the so-called popcorn polymers, which are referred to as “proliferous polymers” or as in the case of polyvinyl pyrrolidone as PVPP.

Within the context of this invention, “branched”, “branching”, “crosslinked”, “crosslinking” are used interchangeably to mean a polymer which has at least one branching site.

“Polymer” also comprises the copolymers, graft homopolymers or graft copolymers, which can in each case be present as linear or soluble-crosslinked, in particular water-soluble-crosslinked, or insoluble-crosslinked, in particular water-insoluble-crosslinked, polymers.

“Polymer” can be present in any form, for example as di- or multi-block polymers, and also be present in star form, brush form or hyperbranched form or as dendrimer.

Polymers according to the invention comprise one or more monomers a), optionally one or more monomers b), and optionally one or more crosslinking monomers c), i.e. they have been obtained by polymerization of the specified monomers and can also comprise residual amounts of the monomers.

Monomers a) are selected from:

N-Vinyllactams such as N-vinylpyrrolidone, N-vinylpiperidone, N-vinylcaprolactam, derivatives thereof substituted with C1 to C8-alkyl groups such as 3-methyl-, 4-methyl- or 5-methyl-N-vinylpyrrolidone and mixtures thereof, vinyl acetate and mixtures of N-vinyllactams, preferably with N-vinylpyrrolidone, vinyl acetate and also acylates and methacylates including acids thereof and monomer mixtures thereof with N-vinyllactams and/or vinylimidazoles and/or quaternized vinylimidazoles, and also diisocyanates and diamines for the formation of polyurethanes with or without terminal EO/PO fatty alcohols, N-vinylamides such as N-vinylformamide and its N-vinylamine obtainable after the polymerization by hydrolysis, N-vinyl-N-methylacetamide.

Amines such as N-vinyl- or allyl-substituted heterocyclic compounds, preferably N-vinylpyridine, or N-allylpyridine, N-vinylimidazoles, which can also be substituted in the 2, 4 or 5 position with C1-C4-alkyl, in particular methyl or phenyl radicals, such as 1-vinylimidazole, 1-vinyl-2-methyl(ethyl)vinylimidazole, and quaternized analogs thereof such as 3-methyl-1-vinylimidazolium chloride, 3-methyl-1-vinylimidazolium methyl(ethyl)sulfate, N-C1- to C24-alkyl-substituted diallylamines or quaternized analogs thereof such as diallylammonium chloride or diallyldimethylammonium chloride and diamines.

Here, polymers according to the invention preferably comprise at least one N-vinyllactam monomer.

Polymers according to the invention can be homopolymers or copolymers of two or more monomers a), for example copolymers of N-vinylpyrrolidone and N-vinylimidazole, copolymers of N-vinylpyrrolidone and N-vinylformamide, copolymers of N-vinylpyrrolidone and N-vinylcaprolactam, copolymers of N-vinylpyrrolidone, N-vinylcaprolactam and N-vinylimidazole or copolymers of N-vinylpyrrolidone and N-vinylimidazole and quaternized N-vinylimidazoles.

Preferred monomers a) are vinyllactams such as N-vinylpyrrolidone, 3-methyl-N-vinyl-pyrrolidone, 4-methyl-N-vinylpyrrolidone, 5-methyl-N-vinylpyrrolidone, N-vinylpiperidone and N-vinylcaprolactam, vinyl acetate, and the vinyl alcohol obtainable after the polymerization by hydrolysis, vinylamides such as vinylformamide, and the vinylamine obtainable by hydrolysis after the polymerization, N-vinylimidazole, 1-vinyl-3-methylimidazolium chloride, 1-vinyl-3-methylimidazolium sulfate, and vinylmethylamide, and derivatives thereof.

Further preferred monomers a) are acylates and methacylates and their acids, and also diamines.

Very particularly preferred monomers a) are N-vinylpyrrolidone, N-vinylcaprolactam, vinyl acetate, vinylformamide, and also the vinylamine obtainable by hydrolysis after the polymerization, and also N-vinylimidazole.

Suitable monomers b) are all monomers which are specified in WO 2010/072640 A1 as “monomer b)” on page 6, from line 8, to page 8, line 17, to which reference is made here in its entirety.

Preferred monomers b) are acrylates, acrylic acid, alkylacrylates, alkylacrylic acids, methacrylates, methacrylic acid, alkyl methacrylates, alkylmethacrylic acids, maleic acid, maleic anhydride, isopropylmethacrylamide, acrylamide, methacrylamide, 2-hydroxyethyl-acrylamide and 2-hydroxyethylmethacrylamide, also vinyl esters of aliphatic C2-C18-carboxylic acids such as vinyl acetate, and also the vinyl alcohol obtainable by hydrolysis after the polymerization, vinyl propionate, vinyl butyrate, vinyl laurate, vinyl stearate, vinyl neodecanoate VEOVA 9 and VEOVA 10, also dimethylaminoethyl (meth)acrylate and dimethylaminoethyl(meth)acrylamide and quaternized analogs thereof, and also diallyldimethylammonium chloride and diisocyanates, and also styrene and styrene derivatives.

Very particularly preferred monomers b) are methacrylamide, vinyl acetate, and the vinyl alcohol obtainable by hydrolysis after the polymerization, vinyl propionate, vinyl neodecanoate VEOVA 9 and VEOVA 10, dimethylaminoethyl (meth)acrylate or dimethyl-aminoethyl(meth)acrylamide or quaternized analogs thereof, and also diallyldimethyl-ammonium chloride.

Polymers which are copolymers and comprise monomers b) can comprise one or more of the monomers b). Usually, however, not more than five different monomers b) are present in one copolymer.

The preferred polymers further include copolymers which comprise one or more monomers a) and/or one or more monomers b).

Suitable crosslinking monomers c) (“crosslinkers”) are:

crosslinking monomers c) are described for example in WO2009/024457 on page 7, line 1 to page 9, line 2, to which reference is expressly made here.

Particularly preferred crosslinking monomers c) are pentaerythritol triallyl ether, methylene-bisacrylamide, N,N′-divinylethyleneurea, divinylbenzene, ethylenebis-N-vinylpyrrolidone, 3-vinyl-N-vinylpyrrolidone, 4-vinyl-N-vinylpyrrolidone, 5-vinyl-N-vinylpyrrolidone, allyl (meth)acrylate, triallylamine and acrylic acid esters of glycol, butanediol, trimethylolpropane or glycerol, and also acrylic acid esters of glycol, butanediol, trimethylolpropane or glycerol reacted with ethylene oxide and/or epichlorohydrin.

The quantitative fractions in percent by weight based on the total mass of the polymer here are, for the monomers a), usually at least 20, preferably at least 30, particularly preferably at least 50, especially preferably at least 60 percent by weight and very particularly preferably up to 100 percent by weight, such as, for example, homopolymers of 100% of a monomer a).

The quantitative fractions in percent by weight, based on the total mass of the polymer, here are, for the monomers b), usually up to 80, preferably up to 70, particularly preferably up to 50, especially preferably up to 40 and very particularly preferably less than 5 percent by weight and are for example not present at all in the polymer.

If the polymer is water-soluble crosslinked polymer, the quantitative fractions of the crosslinking monomers c) in percent by weight, based on the total mass of the polymer, are usually 0.001 to 20 percent by weight, preferably 0.01 to 10 percent by weight, particularly preferably 0.05 to 5 percent by weight and in particular 0.1 to 1 percent by weight.

If the polymer is water-insoluble crosslinked polymer such as, for example, a popcorn polymer, the quantitative fractions of the crosslinking monomers c) in percent by weight, based on the total mass of the polymer, are usually 0.001 to 10, preferably 0.01 to 5, particularly preferably 0.1 to 3 and especially 0.5 to 2 percent by weight.

If crosslinking monomer c) is used, then the overall quantitative fractions of monomer a) and optionally monomer b) are reduced correspondingly by the amount of crosslinking monomer c) used.

The monomers a), b) and c) used for the polymerization can, independently of one another, be an individual or mixtures of two or more monomers a), monomers b) and/or monomers c), where the combined quantitative fraction of the monomers a), b) or c) gives the quantitative fraction specified in each case therefore for monomer a), for monomer b) or for monomer c) based on the polymer.

The total amounts of monomer(s) a) plus monomer(s) b) plus monomer(s) c) always add up here to 100 percent by weight.

A vinyllactam polymer can be a homopolymer or copolymer comprising N-vinyllactams such as N-vinylpyrrolidone (VP) or 3, 4- or 5-position methyl-substituted derivatives thereof, N-vinylpiperidone or N-vinylcaprolactam (VCap). Preference is given to N-vinylpyrrolidone, N-vinylcaprolactam or mixtures thereof. N-Vinylpyrrolidone is especially preferred. Preferred vinyllactam polymers are vinylpyrrolidone polymers such as polyvinylpyrrolidones, vinylpyrrolidone copolymers and vinylpyrrolidone popcorn polymers.

Preferred polyvinylpyrrolidones are polymers with K values of 1 to 150, preferably K10 to K120, for example K12, K15, K17, K25, K30, K60, K85, K90, K95, K100, K115 or K120.

Particularly preferred PVP homopolymers have a K value of 12 to 95 and especially preferably of 30 to 90, such as in particular K30, K60, K85 and K90.

Preferred vinylpyrrolidone copolymers are linear, uncrosslinked copolymers with N-vinylcaprolactam (VCap), vinyl acetate (VAc), N-vinylimidazole (VI), quaternized N-vinylimidazole and/or derivatives thereof and/or mixtures thereof.

Particularly preferred copolymers are copolymers of N-vinylpyrrolidone (VP) with vinyl acetate with a weight ratio VP/VAc of 20:80 to 80:20, for example 30:70, 50:50, 60:40, 70:30, with K values of 10 to 90, preferably of 15 to 80 and in particular of 20 to 60. Very particularly preferred copolymers of N-vinylpyrrolidone with vinyl acetate have a K value of 22 to 40 and a weight ratio VP to VAc of 50:50 to 70:30.

Preference is likewise given to copolymers of VP and VCap with K values of 10 to 100, preferably of 12 to 80 and in particular of 20 to 75, and also weight ratios of the monomers VP to VCap from 80:20 to 20:80, preferably from 70:30 to 30:70, especially preferably from 60:40 to 40:60 and for example also 50:50.

The K value of the vinylpyrrolidone copolymers and of the polyvinylpyrrolidones (Fikentscher K value; see for example Buhler, “Polyvinylpyrrolidone—Excipient for Pharmaceuticals”, Springer, 2005, pages 40 to 41) is a measure of the solution viscosity at defined conditions. Consequently, it is a direct measure of the molar mass. If the molar mass changes, for example as a result of oxidative processes, this leads to an increase in molar mass (leads to an increase in K value) or to a reduction in molar mass (leads to a reduction in K value) and thus to a change in the K value. If the molar mass changes, then the solution viscosity of a solution with a defined solids content also changes accordingly.

Preferred aqueous polymer solutions are polyvinylpyrrolidone solutions, polyvinylpyrrolidone/polyvinyl acetate solutions and polyurethane solutions.

The aqueous polymer solutions are prepared by conventional processes, for example by stirring in the preservative mixtures according to the invention or by directly mixing in the individual components of the three specified preservatives/boosters.

The stabilized polymers of the invention are prepared from the aqueous solutions in a customary manner, for example by drying, such as spray drying, drying on contact surfaces and drying by means of vacuum.

According to the invention, the aqueous polymer solutions stabilized with the preservative mixture and the polymers thereof are used in cosmetic preparations as, for example, binders, thickeners or film formers. Examples of such cosmetic preparations are skincare and hair care compositions. The skincare compositions include, for example, creams, gels, gel creams, waxes, wax gels, gel waxes and lotions. The hair care compositions include, for example, creams, gels, gel creams, waxes, wax gels, gel waxes, styling and conditioning foams, and also spays (pump sprays and aerosol sprays) and lotions. In this connection, mention is also to be made of products for cosmetic cleaning, which include shower gels and bath gels.

The stabilized aqueous polymer solutions according to the invention and the polymers therefrom can likewise be used in decorative cosmetic. These include, for example, make-ups, lipsticks, concealers and mascaras.

A cosmetic preparation is usually in the form of a gel, foam, spray or emulsion and can comprise a number of further cosmetic additives. Depending on the use of the preparation according to the invention, further additives also have to be admixed; these are selected from the group which is formed from emulsifiers, pearlescent waxes, stabilizers, salt, thickeners, consistency regulators, inorganic and organic UV photoprotective filters, self-tanning agents, pigments, antioxidants, hydrotopes, biogenic active ingredients, dyes, customary preservatives, preferably benzoic acid or citric acid, humectants such as glycerol, ethanol, propylene glycol, antidandruff agents, swelling agents and perfumes. Preferred biogenic active ingredients here are in particular tocopherol, tocopherol acetate, tocopherol palmitate, deoxyribonucleic acid, coenzyme Q10, ascorbic acid, retinol and retinyl derivatives, bisabolol, allantoin, phytantriol, panthenol, AHA acids, amino acids, ceramides, essential oils, hyaloronic acid, creatine, protein hydrolysates, plant extracts, peptides and vitamin complexes.

A further aspect of the present invention lies in the use of the aqueous polymer solution stabilized according to the invention or of the polymers thereof as conditioning agents in hair care compositions. The aqueous solution stabilized according to the invention and the polymers thereof can be incorporated into hair shampoos, hair care compositions, hair lotions, hair treatments, hair gel, hair mousses, conditioners, hair tonics and hairstyling products, such as styling and conditioning mousses, hair gels, hair mousses, hair-setting compositions (in e.g. sprays and lotions) and hair waxes. The use in hair shampoo necessitates the use of further surfactants and also auxiliaries and additives. The addition of further customary additives depends on the particular product.

On account of its biocidal properties and its use in extremely low concentrations, the preservative mixture according to the invention is exceptionally suitable for stabilizing aqueous polymer solutions. The product stability of the stabilized polymer solutions is retained over a long period, the appearance and the sensory properties are unchanged and there are no color changes and clouding, nor phase separation of the solutions. The polymer solutions according to the invention and also the polymers thereof exhibit an extraordinarily good microbial stability and can be incorporated without concern into cosmetic products and hair care compositions and also hairstyling products.

The invention is illustrated by reference to the following examples.

### EXAMPLES

### Example 1

An aqueous polyvinylpyrrolidone (PVP) solution (Luviskol K 85L, 20% strength aqueous solution) was admixed with a preservative mixture of phenoxyethanol (Protectol PE), ethylhexylglycerol (Sensiva SC 50) and 2-methylisothiazolinone (Microcare MT). The concentration of the constituents in the PVP solution was 0.001% 2-methylisothiazolinone, 0.1% ethylhexylglycerol and 0.5% phenoxyethanol.

The germ loading test according to Ph. Eur., 7th Edition with the stabilized PVP solution exhibited, after 28 days, freedom from germs with regard to the tested strains Pseudomonas aeruginosa (ATCC 9027), Staphylococcus aureus (ATCC 6638), Candida albicans (ATCC 10231) and Aspergillus brasiliensis (ATCC 16404).

Furthermore, the even more stringent “KoKo test” (KoKo test SM 021) was readily satisfied following repeated inoculation.

The stability test of the PVP solution has revealed that the PVP solution was still clear and colorless after storage for 12 weeks. No change in odor was recorded.

### Comparative Example 1

An aqueous polyvinylpyrrolidone (PVP) solution (Luviskol K 85L, 20% strength aqueous solution) was admixed with a preservative mixture of ethylhexylglycerol and phenoxyethanol (Euxyl PE 9010). The concentration of the constituents in the PVP solution was 0.05% ethylhexylglycerol and 0.45% phenoxyethanol.

The germ loading test according to Ph. Eur., 7th Edition was not passed. Although it revealed, after 28 days, freedom from germs with regard to the tested strains Pseudomonas aeruginosa (ATCC 9027), Staphylococcus aureus (ATCC 6638) and Candida albicans (ATCC 10231), a colonization with Aspergillus brasiliensis (ATCC 16404) was established. The KoKo test was not passed, with severe mold formation being exhibited after just one inoculation cycle.

The stability test with a hair gel ready-to-use formulation exhibited a yellow coloration with the products after 3 months.

### Example 2

An aqueous polyurethane (PUR) solution (Luvigel Star, an alkoxylated polyurethane terminally modified with fatty alcohols) was admixed with a preservative mixture of ethylhexylglycerol (Sensiva SC 50), 2-methylisothiazolinone (Microcare MT) and phenoxyethanol (Protektol PE). The concentration of the constituents in the polyurethane solution was 0.2% ethylhexylglycerol, 0.01% 2-methylisothiazolinone and 0.5% phenoxy-ethanol.

The germ loading test according to Ph. Eur., 7th Edition with the stabilized PUR solution revealed, after 28 days, freedom from germs with regard to the tested strains Pseudomonas aeruginosa (ATCC 9027), Staphylococcus aureus (ATCC 6638), Candida albicans (ATCC 10231) and Aspergillus brasiliensis (ATCC 16404).

In addition, the even more stringent “KoKo test” (KoKo test SM 021) was readily satisfied following repeated inoculation.

### Comparative Example 2

An aqueous polyurethane (PUR) solution (Luvigel Star, an alkoxylated polyurethane terminally modified with fatty alcohols) was admixed with a preservative mixture of ethylhexylglycerol and phenoxyethanol (Euxyl PE 9010). The concentration of the constituents in the polyurethane solution was 0.125% ethylhexylglycerol and 1.125% phenoxyethanol. Even at these very high feed substance concentrations of the aforementioned preservatives, although freedom from germs with regard to the tested strains Pseudomonas aeruginosa (ATCC 9027), Staphylococcus aureus (ATCC 6638) and Candida albicans (ATCC 10231) was established in the germ loading test according to Ph. Eur., 7th

Edition after 28 days, it was not established with regard to Aspergillus brasiliensis (ATCC 16404). Criterion A and B according to Ph. Eur. 7th Edition were not satisfied.

The results from the KoKo test also revealed that the solution had a considerable colonization of mold even after the first inoculation cycle.

Furthermore, these polymer solutions with the preservative mixture from comparative example 2 exhibit product instabilities with regard to clouding/phase separation, discolorations and intensive odors after storage at various temperatures (6° C., RT, 40° C.), and also negative performance properties, such as declines in the emulsion and gel viscosities.

### Example 3

An aqueous polyvinylpyrrolidone (PVP) solution (Luviskol K 90L, 20% strength aqueous solution) was admixed with a preservative mixture of phenoxyethanol (Protectol PE), ethylhexylglycerol (Sensiva SC 50) and 2-methylisothiazolinone (Microcare MT). The concentration of the constituents in the PVP solution was 0.004% 2-methylisothiazolinone, 0.04% ethylhexylglycerol and 0.5% phenoxyethanol.

The germ loading test according to Ph. Eur., 7th Edition with the stabilized PVP solution exhibited, after 28 days, freedom from germs with regard to the tested strains Pseudomonas aeruginosa (ATCC 9027), Staphylococcus aureus (ATCC 6638), Candida albicans (ATCC 10231) and Aspergillus brasiliensis (ATCC 16404).

Furthermore, the even more stringent “KoKo test” (KoKo test SM 021) was completely satisfied following inoculation six times (in each case freedom from germs after inoculation 6 times).

### Comparative Example 3

An aqueous polyvinylpyrrolidone (PVP) solution (Luviskol K 90L, 20% strength aqueous solution) was admixed with a preservative mixture of ethylhexylglycerol and phenoxyethanol (Euxyl PE 9010). The concentration of the constituents in the PVP solution was 0.05% ethylhexylglycerol and 0.45% phenoxyethanol.

The germ loading test according to Ph. Eur., 7th Edition was not passed. Although it revealed, after 28 days, freedom from germs with regard to the tested stains Pseudomonas aeruginosa (ATCC 9027) and Staphylococcus aureus (ATCC 6638), only a limited freedom from germs was recorded for Candida albicans (ATCC 10231). However, a colonization with Aspergillus brasiliensis (ATCC 16404) was established. The KoKo test was also not passed, there being a severe colonization of yeasts and molds even after one inoculation cycle.

### Comparative Example 4

An aqueous polyvinylpyrrolidone (PVP) solution (Luviscol K90L, 20% strength aqueous solution) was admixed with the preservative phenoxyethanol (Protectol PE). The concentration of phenoxyethanol in the PVP solution was 1%.

The germ loading test according to Ph. Eur., 7th Edition was not passed. A severe Aspergillus brasiliensis colonization was produced after 28 days. The even more stringent Koko test exhibits a considerable colonization of bacteria, molds and yeasts even after the first inoculation cycle.

All comparative examples with concentrations of 0.75-1% Euxyl PE 9010 (ethylhexylglycerol and phenoxyethanol) or 0.1-0.2% Euxyl K 220 (ethylhexylglycerol and phenoxyethanol) exhibit product instabilities with regard to clouding, discolorations, high odor intensities after storage or unacceptable performance properties, such as clouding, discolorations, viscosity decline for formulations, such as e.g. gels.

1. An aqueous polymer solution stabilized with a preservative mixture that comprises:
0.01 to 0.2% by weight of ethylhexylglycerol,
0.001 to 0.01% by weight of 2-methylisothiazolinone, and
0.05 to 0.5% by weight of phenoxyethanol.

2. The aqueous polymer solution according to claim 1, which comprises:
0.04 to 0.15% by weight of ethylhexylglycerol,
0.003 to 0.008% by weight of 2-methylisothiazolinone, and
0.1 to 0.5% by weight of phenoxyethanol.

3. The aqueous polymer solution according to claim 1, which comprises:
0.04±0.004% by weight of ethylhexylglycerol,
0.0038±0.0004% by weight of 2-methylisothiazolinone, and
0.5±0.05% by weight of phenoxyethanol.

4. The aqueous polymer solution according to claim 1, wherein the polymer is selected from the group consisting of: vinyllactam polymers, polyethers, polyalkylenimines, polyvinylamines, polyvinylformamides and partially hydrolyzed products thereof, polyimides, polyamides, and polyurethanes, and mixtures thereof.

5. The aqueous polymer solution according to claim 4, wherein the polymer is selected from the group consisting of: N-vinylpyrrolidone, N-vinylcaprolactam, vinyl acetate, vinylformamide, polyurethane, and the vinylamine obtainable by hydrolysis after the polymerization or N-vinylimidazole, quaternized vinylimidazoles, styrene, and styrene derivatives.

6. A preservative mixture comprising the following constituents as biocidal active ingredients:
62.5% by weight of phenoxyethanol,
25.0% by weight of ethylhexylglycerol, and
1.25% by weight of 2-methylisothiazolinone, and
water as remainder.

7. The aqueous polymer solution according to claim 1 further comprising 0.08 to 0.8% by weight of a preservative mixture comprising biocidal active ingredients: 62.5% by weight of phenoxyethanol; 25.0% by weight of ethylhexylglycerol; 1.25% by weight of 2 methylisothiazolinone; and water as remainder.

8. A preservative mixture comprising the following constituents as biocidal active ingredients
86. 20% by weight of phenoxyethanol,
6.90% by weight of ethylhexylglycerol,
0.69% by weight of 2-methylisothiazolinone, and
water as remainder.

9. The aqueous polymer solution according to claim 3 further comprising 0.4 to 0.6% by weight of a preservative mixture comprising as biocidal active ingredients: 86.20% by weight of phenoxyethanol; 6.90% by weight of ethylhexylglycerol; 0.69% by weight of 2 methylisothiazolinone; and water as remainder.

10. A polymer obtained from an aqueous solution according to claim 1.

11. The polymer according to claim 10, obtained by drying.

12. A method of preparing a cosmetic preparation, the method comprising: obtaining the aqueous polymer solution according to claim 1, and mixing the aqueous polymer solution with one or more cosmetic ingredients to form a cosmetic preparation.

13. The method according to claim 12, wherein the cosmetic preparation comprises a skincare or a hair care composition.

14. The method of claim 13, wherein the hair care composition comprises a hairstyling product.

15. The method of claim 12 wherein the cosmetic preparation comprises a decorative cosmetic or a hair and body composition.

16. A method of preparing a cosmetic preparation, the method comprising: obtaining the polymer according to claim 10, and mixing the polymer with one or more cosmetic ingredients to form a cosmetic preparation.

17. The method according to claim 16, wherein the cosmetic preparation comprises a skincare or a hair care composition.

18. The method of claim 17, wherein the hair care composition comprises a hairstyling product.

19. The method of claim 16 wherein the cosmetic preparation comprises a decorative cosmetic or a hair and body composition.
